(19) United States
(12) Patent Application Publication
(10) Pub. No.: US 2006/0160100 A1
(43) Pub. Date: Jul. 20, 2006

(54) Enzymatic electrochemical detection assay using protective monolayer and device therefor

(75) Inventors: Zhiqiang Gao, Singapore (SG); Ponnampalam Gopalakrishnakone, Singapore (SG); Van Dong Le, Singapore (SG); Fang Xie, Singapore (SG)

Correspondence Address: KLARQUIST SPARKMAN, LLP, 121 SW SALMON STREET, SUITE 1600, PORTLAND, OR, 97204, US

(73) Assignees: Agency for Science, Technology and Research; National University of Singapore

(21) Appl. No.: 11/038,289

(22) Filed: Jan. 19, 2005

(51) Int. Cl. C12Q 1/68; C12Q 1/00

(52) U.S. Cl. 435/6; 205/777.5

(57) ABSTRACT

There is provided an electrochemical assay method for detecting a target molecule, for example a protein, in a sample, which involves the use of a protective monolayer and a redox polymer to form a bilayer immobilized on an electrode. The monolayer protects the electrode from non-specific adherence of reagents, particular proteins, to the electrode while simultaneously providing a surface that can be functionalized to immobilize a capture molecule and that can interact with the redox polymer.

### FIELD OF THE INVENTION

The present invention relates generally to assays using electrochemical detection methods, and devices for practising same.

### BACKGROUND OF THE INVENTION

Electrochemical immunoassays have been developed as methods for detection of proteins in a sample. Such assays have been developed as an alternative to radioassays, fluorometric or colourimetric assays, due to the ease of detecting electrochemically active molecules, and the elimination of the need for specialized and complicated detection devices. Electrodes used in detection of the electrochemically active molecules can be miniaturized for inclusion in portable devices for point-of-care and field uses. Furthermore, the electrodes can be easily arranged into microarray platforms for multiplexing applications.

One method of electrochemical detection is by amperometric assay, which entails measuring current flow which results from a redox reaction. Amperometric measurements allow for rapid detection of electrochemically active species, and have a broad linear range and low detection limit, for example, as low as 10⁻¹⁰ A for some methods.

Several different approaches to amperometric electrochemical protein detection assays have been developed. Generally, electrochemical protein detection assays couple the detection of an analyte protein with enzyme-catalyzed electron transfer to or from a substrate, and ultimately to a half-cell reaction of an electrochemical cell. Since the enzyme catalyzed oxidation or reduction of substrate is coupled with the redox reaction of the electrode, most assay methods involve either delivering to the vicinity of the electrode the enzyme which catalyzes the electron transfer reaction, or conversely blocking such delivery, when the analyte protein is present in solution. In the case of protein detection, the analyte protein may be captured using an anti-analyte antibody.

Assays that rely on blocking the delivery of the enzyme are often performed competitively. Typically, the enzyme used in the electron transfer redox reaction is first conjugated to a competitor of the analyte protein (often purified analyte protein) prior to conducting the assay. An anti-analyte antibody that recognizes both analyte and competitor is immobilized at or near the electrode. Analyte is added, which is captured by the anti-analyte antibody, followed by the conjugated competitor-enzyme. When the competitor-bound enzyme is captured by anti-analyte antibody, the enzyme is available to reduce or oxidize a substrate in the vicinity of the electrode. The electrode surface is typically washed between steps to remove excess reagents from each step of the assay to minimize signal that is derived from non-specifically bound reagents. The greater the concentration of analyte in the sample, the more analyte binds to the antibody, resulting in lesser amounts of competitor-enzyme available to catalyze an electron transfer in the vicinity of the electrode surface. The binding of analyte is thereby indicated by the level of the enzyme catalyzed electron transfer reaction, which is in turn coupled with the redox reaction of the electrode.

One drawback of the competitive assay is that the concentration of the analyte protein in the sample is inversely proportional to the amount of current produced by reduction or oxidation at the electrode, and such assays are therefore not very sensitive and have a limited detection range.

As mentioned, the above-described competitive assays require the separation of unbound protein reagents, such as unbound analyte or excess competitor-enzyme, by exchanging the solution that is in contact with the electrode in washing steps. For example, excess competitor enzyme that is not bound by anti-analyte antibody can contribute to a falsely high signal in the assay. Such washing of the surface at which the various reagents are being added (usually the electrode surface), typically after addition of each reagent such as analyte, competitor (or competitor-enzyme), or anti-analyte antibody, adds to the complexity of the assay. The washing steps are important to minimize non-specific binding of proteins to the electrode, and to lower signal from competitor-enzyme which has been excluded from the antibody due to the presence of the antibody.

Separation-free competitive assays, which do not require the separation steps, have been developed for in-field use, in which the above principles have been adapted to a one-step process.

One separation-free method involves the use of a layer of immobilized competitor and bound anti-analyte antibody on the electrode surface to block access of free enzyme to the electrode. Briefly, the electrode surface is modified with an immobilized competitor and an electron transfer mediator. The mediator is typically a redox-active molecule that assists in transfer of electrons from the enzyme active site to the electrode. Sample is added, which may contain analyte, followed by anti-analyte antibody. In the absence of analyte, the anti-analyte antibody binds to the immobilized competitor, forming a blocking layer at the electrode surface. When analyte is present, the anti-analyte antibody will bind to the analyte in solution and be prevented from forming the layer at the electrode surface. Free enzyme is added to the solution, which catalyzes a redox reaction. The enzyme requires interaction with the mediator in order to exchange electrons with the electrode, which exchange results in detectable current flow. The layer, when formed, prevents the free enzyme from interacting with the mediator bound to the electrode. This method relies on the formation of a high quality competitor/anti-analyte antibody layer and is very sensitive to defects within such layer. Such competitor/anti-analyte antibody layers may be difficult to produce since the direct binding of competitor protein to the surface can result in random orientation of the competitor protein on the electrode surface and sub-optimal binding of the antibody to form the competitor/anti-analyte antibody layer due to the random positioning of relevant epitopes on the surface of the competitor protein.

Lu et al. (Anal. Chim. Acta (1997) 345: 59-66; Anal. Comm. (1997) 34: 21-24) describe an “electrically wired” approach. An electrode is coated with immobilized anti-analyte antibody and a redox polymer, which acts as a mediator between the electrode and the enzyme that is used in the assay. Sample is added, and any analyte in the sample will bind to the anti-analyte antibody. Competitor-bound enzyme is brought to the electrode surface through capture by anti-analyte antibody which is not bound to analyte, at a concentration that it inversely proportional to the amount of analyte already bound. Enzyme substrate is added and is reduced or oxidized by the enzyme attached to the captured competitor. The redox polymer regenerates the active site of the captured enzyme through electron transfer, transferring electrons to or from the electrode and allowing for detection of the electron transfer redox reaction. However, as with the above-described approaches, immobilization of the antibody onto the electrode surface is difficult and can result in denaturation or sub-optimal orientation or concentration of the antibody.

As well, in the Lu assay, non-specific binding of the conjugated competitor-enzyme at the electrode coated with anti-analyte antibody and redox polymer can result in incorrectly low measurements of analyte concentration due to an increase in signal derived from the non-specific binding. Generally, proteins often adhere non-specifically to surfaces, including electrode surfaces. In separation-free electrochemical assays, this effect may be heightened since no washing step is involved. Where there is such non-specific adherence of conjugated competitor-enzyme, the assay will detect signal through the electrode that is not dependent on the presence of analyte, resulting in false readings, and increasing the detection limit of the assay.

Thus, there exists a need for an electrochemical detection assay with high sensitivity and low detection limits, which minimizes non-specific, non-analyte mediated interaction of the redox enzyme with the electrode.

### SUMMARY OF THE INVENTION

In one aspect, there is provided a method of electrochemically detecting a target molecule in a sample, comprising: coating an electrode with a monolayer capable of immobilizing a capture molecule thereon and of transferring electrons there across, thereby inhibiting non-specific binding of protein molecules at the electrode; immobilizing the capture molecule on the monolayer; adding a sample containing a target molecule to be captured by the capture molecule; adding a detection molecule that specifically binds one of the capture molecule or the target molecule, wherein the detection molecule is labelled with an enzyme capable of oxidizing or reducing a substrate; adding a redox polymer that interacts with the monolayer and that together with the monolayer forms a conductive path from the enzyme to the electrode; adding the substrate to be oxidized or reduced by the enzyme; and detecting current flow at the electrode.

In another aspect, there is provided a method of providing an electrochemical cell for detecting a target molecule in a sample, comprising: coating an electrode with a monolayer capable of immobilizing a capture molecule thereon and of transferring electrons there across, thereby inhibiting non-specific binding of protein molecules at the electrode; and immobilizing the capture molecule thereon.

In a further aspect, there is provided a device for performing an electrochemical assay, comprising: a monolayer formed on a surface of an electrode, the monolayer capable of immobilizing a capture molecule thereon and of transferring electrons there across, thereby inhibiting non-specific binding of protein molecules at the electrode; and a capture molecule immobilized on the monolayer. The device may further comprise a target molecule captured by the capture molecule; a detection molecule specifically bound to one of the capture molecule or the target molecule, wherein the detection molecule is labelled with an enzyme capable of oxidizing or reducing a substrate; and a redox polymer forming an interaction with the monolayer and that together with the monolayer forms a conductive path from the enzyme to the electrode.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention,

FIG. 1 is a schematic diagram of an electrochemical cell used in the present method;

FIG. 2 is a schematic diagram of a modified surface of an electrode on which the present method is performed;

FIG. 3 is a schematic diagram depicting the formation of an analyte layer on the electrode surface;

FIG. 4 is a schematic diagram of a redox polymer assembled on the analyte layer on the electrode surface to form a bilayer;

FIG. 5 is a schematic diagram depicting the conversion of substrate from a first oxidation state to a second oxidation state by an enzyme contained in the bilayer on the electrode surface;

FIG. 6 is a cyclic voltammogram of an electrode coated with a mercapto undecanoic acid monolayer and redox polymer in phosphate buffered saline (PBS), scanned at 100, 200, 300, 400 and 500 mV/s (innermost curve to outermost curve, respectively);

FIG. 7 is a cyclic voltammogram obtained using the present method to detect the presence of test protein β-BuTx (dashed line, no substrate; solid line, with 2.0 mM H₂O₂);

FIG. 8 is a graph depicting the amperometric response of electrodes with test protein, detection antibody and enzyme (trace 1), or BSA control (trace 2).

FIG. 9 is a graph indicating the amperometric response of electrodes with varying amounts of osmium-containing redox polymer; and

FIG. 10 is a titration curve indicating the amperometric response as dependent on varying concentrations of test protein β-BuTx (toxin).

### DETAILED DESCRIPTION

There is presently provided an electrochemical assay method of detecting a target molecule, including a protein, in a sample, which involves the use of a protective monolayer and a redox polymer to form a bilayer immobilized on an electrode. The monolayer protects the electrode from non-specific adherence of protein reagents to the electrode while simultaneously providing a surface that can be functionalized to immobilize a capture molecule and to interact with the redox polymer. The electrode provides a conducting surface with which to monitor electron transfer to or from a substrate, catalyzed by an enzyme, whereby the concentration of enzyme, and thus the extent of electron transfer, is dependent on the concentration of a target molecule in a sample.

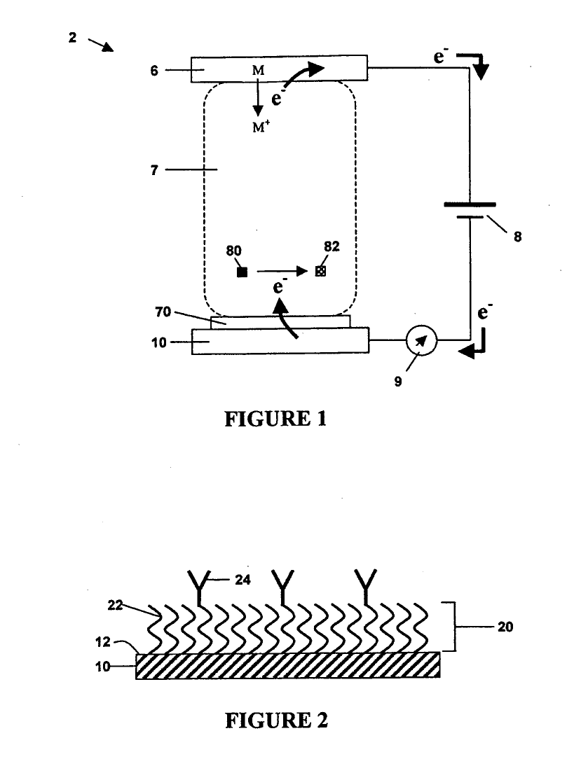

The method may be embodied in an exemplary electrochemical cell 2, as depicted in FIG. 1. A redox reaction catalyzed by an enzyme that reduces or oxidizes a substrate is detected using the electrochemical cell 2, which has a reference electrode 6 and a working electrode 10, each of which is connected to a biasing source 8. An ammeter 9 is also connected in line, to enable measurement of current flow. Reference electrode 6 and working electrode 10 are both in contact with solution 7. In the depicted embodiment, the reference electrode 6 is an anode, and an oxidation reaction takes place at the anode in order for current to flow: metal atoms (M) of the reference electrode give up electrons to become metal ions (M⁺) in solution 7. Solution 7 will contain a supporting electrolyte for neutralization of charge build up in solution 7 at each of electrodes 6 and 10. A reduction reaction takes place at working electrode 10, which in the depicted embodiment is a cathode. The electrons are transferred from electrode 10 via a reduction/oxidation cascade of bilayer 70, which contains a monolayer, a capture antibody, an analyte protein, a detection antibody conjugated to an enzyme and a redox polymer, as is described in greater detail below. The reduction/oxidation cascade of bilayer 70 leads to the eventual reduction of substrate 80 to product 82 in solution 7, which is dependent on the presence of a target analyte to bring the enzyme into bilayer 70. In order to initiate the oxidation and reduction reactions occurring at electrodes 6 and 10, respectively, a potential difference is applied by biasing source 8. A current can flow between reference electrode 6 and working electrode 10, depending on the levels of enzyme catalyzed reduction of substrate 80.

Electrode 10 may be composed of any electrically conducting material, including carbon paste, carbon fiber, graphite, glassy carbon, any metal commonly used as an electrode such as gold, silver, copper, platinum or palladium, a metal oxide such as indium tin oxide, or a conductive polymeric material, for example poly(3,4-ethylenedioxythiophene) (PEDOT) or polyaniline.

Formation of bilayer 70 on surface 12 of electrode 10 is illustrated in FIGS. 2-4. As illustrated in FIG. 2, a functionalized monolayer 20 of a monolayer component molecule 22 is formed on a surface 12 of electrode 10.

The functionalized monolayer 20 is a single layer comprising monolayer component molecule 22. Monolayer component molecule 22 allows for transfer of electrons between the redox polymer and the electrode surface, and may be conductive or non-conductive, provided that if non-conductive, electrons can tunnel across it, and therefore non-conductive monolayer component molecule 22 should be short enough to allow such tunnelling. For example, non-conductive monolayer component molecule 22 may have a backbone of 1 to 20 atoms.

Monolayer component molecule 22 has a hydrophobic region, for example, an aliphatic region, which enables it form a monolayer. Monolayer component molecule 22 further has an end functional group at one end, which is capable of interacting with a complementary functional group on another molecule. For example, the end functional group may be a charged group such as a carboxyl group or an amino group, which is capable of forming an electrostatic interaction with an oppositely charged functional group. Alternatively, the end functional group may be ligand or an affinity group. The ligand or affinity binding molecule may be any molecule that interacts with another molecule through a specific interaction, such as either half of a receptor/ligand pair which bind to each other through a specific, non-covalent affinity interaction. For example, the affinity binding molecule may be biotin, streptavidin, avidin, an ATP analogue, an ATP binding domain, imidazole, digoxigenin or a 6-histidine peptide. When monolayer component molecule 22 is assembled in monolayer 20, the end functional group is positioned at the outer surface of the monolayer, providing a functionalized surface to monolayer 20.

Although in the depicted embodiment the monolayer comprises a single type of monolayer component molecule, in other embodiments the monolayer may comprise two or more types of monolayer component molecules, one of which possesses an end functional group that can interact with and immobilized the capture molecule, and one of which possesses a different end functional group that is capable of interacting with a complementary functional group on the redox polymer.

The portion of monolayer component molecule 22 in contact with electrode 10 may have an electrode-reactive group that reacts with the electrode surface 12, allowing for immobilization of the monolayer component molecule 22 on the electrode surface 12. For example, if the electrode 10 is metal, such as gold, the monolayer component molecule 22 may have a reactive thiol group at the opposite end of the molecule from the end functional group, so as to form a sulfur-gold bond with the gold surface. In certain embodiments, the monolayer component molecule 22 is mercaptoundecanoic acid or mercaptohexadecanoic acid.

Monolayer 20 may be formed on surface 12 by contacting the electrode surface 12 with the monolayer component molecule 22. The monolayer 20 may be formed by self-assembly. For example, the electrode 10 may be immersed in a solution of monolayer component molecule 22 dissolved in a suitable organic solvent. The organic solvent is any solvent in which monolayer component molecule 22 is soluble, and may be, for example, ethanol, tetrahydrofuran, chloroform, dichloromethane, 1,2-dichloroethane, 1,1,2,2-tetrachloroethane, toluene, xylene, chlorobenzene, 1,2-dichlorobenzene, cyclohexanone or 2-methylfuran. Upon immersion of the electrode 10, monolayer component molecule 22 will arrange itself on the surface 12 of electrode 10, with the end functional group free in solution, and if applicable, with the electrode-reactive group at the electrode surface 12. Alternatively, Langmuir-Blodgett techniques, and other methods known in the art as describe in Yang et al. (1999) J. Electroanalytical Chem. 470: 114-119; Gao et al., (1995) Synthetic Metals 75: 5-10; and Swalen et al. (1987) Langmuir 3: 932-950; Gao et al. (1997) Electrochimica Acta 42: 315-321, may be used to form a monolayer 20 of monolayer component molecule 22 on electrode surface 12.

Once formed, the functionalized monolayer 20 is preferably uniform and pinhole free, meaning that no gaps exist, thereby preventing solution molecules from contacting electrode surface 12. However, in the event that any defects are present in the monolayer, the redox polymer, when layered as described below, may be able to penetrate the monolayer to contact the surface 12 of electrode 10. The monolayer should be such that any defects or pinholes that do exist are not large enough to permit bulky protein molecules, particularly the enzyme used to catalyze electron transfer, to access the surface 12 of electrode 10. Thus, when electrode surface 12 is coated with a monolayer comprising monolayer component molecule 22, molecules in a bulk solution which may be deposited on surface 12 cannot directly contact the electrode surface, but instead interact with the functionalized surface of monolayer 20, since the functionalized monolayer 20 forms a protective layer on electrode surface 12. Non-specific interactions at the electrode surface of the enzyme used to catalyze the electron transfer reaction are therefore significantly reduced or inhibited, and thus should not significantly contribute to the amperometric signal detected at electrode 10. A non-specific interaction, or non-specific binding refers to an interaction or binding of a molecule with another molecule or surface which does not distinguish the other molecule or surface over other binding partners, or which is not based on a chemical affinity or selective interaction.

Once monolayer 20 has been formed, capture antibody 24 is immobilized at the surface of functionalized monolayer 20 as illustrated in FIG. 2. This may be achieved by modifying the end functional group of monolayer component molecule 22 to allow for binding with, and immobilization of, capture antibody 24. An end functional group may be modified, for example, by reacting with a bi-functional cross-linking molecule which also reacts with particular functional groups within capture antibody 24, thereby immobilizing capture antibody 24 at the surface of monolayer 20. Alternatively, the end functional group of monolayer component molecule 22 may be able to react with or interact with a particular complementary functional group contained within capture antibody 24, thereby binding and immobilizing capture antibody 24 directly. Capture antibody 24 is immobilized at the surface of monolayer 20, meaning that capture antibody 24 is bound to monolayer 20 via an interaction between capture antibody 24 and the end functional group of monolayer component molecule 22.

Capture antibody 24 may be immobilized on the functionalized monolayer 20 by contacting a solution containing capture antibody 24 with surface 12, to covalently bind capture antibody 24 to the functionalized monolayer 20 through the functional groups on monolayer component molecule 22. Alternatively, capture antibody 24 may be cross-linked to the end functional groups, using standard cross-linking methods known in the art. Generally, a cross-linker will have two reactive groups, which may be the same or different, and which are available to react with the end functional group on monolayer component molecule 22 and with groups within capture antibody 24. For example, where the end functional group is a carboxylic group, it may be cross-linked to amino groups in capture antibody 24 using standard 1-ethyl-3-(3-dimethylaminopropyl)carbodiimide/N-hydroxysuccinimide (EDC/NHS) methods. A skilled person will appreciate that the method of immobilizing capture antibody 24 should not disrupt the ability of capture antibody 24 to bind its target protein 46.

Capture antibody 24 is immobilized on functionalized monolayer 20 at a density such that functionalized monolayer 20 retains accessible end functional groups at its surface. In most embodiments, capture antibody 24 will be a bulkier molecule than monolayer component molecule 22, meaning that the ratio of capture antibody 24 to monolayer component molecule 22 will be less than 1 due to steric hindrance between adjacent molecules of capture antibody 24. Additionally, a skilled person will be able to readily adjust the concentration of capture antibody 24, and any crosslinker used to immobilize capture antibody 24 on functionalized monolayer 20 so as to achieve a desired density. Thus, even though capture antibody 24 is attached to monolayer 20 via the end functional group of monolayer component molecule 22, sufficient available end functional groups remain at the surface of functionalized monolayer 20 to be available for interaction with a redox polymer 60 as described below.

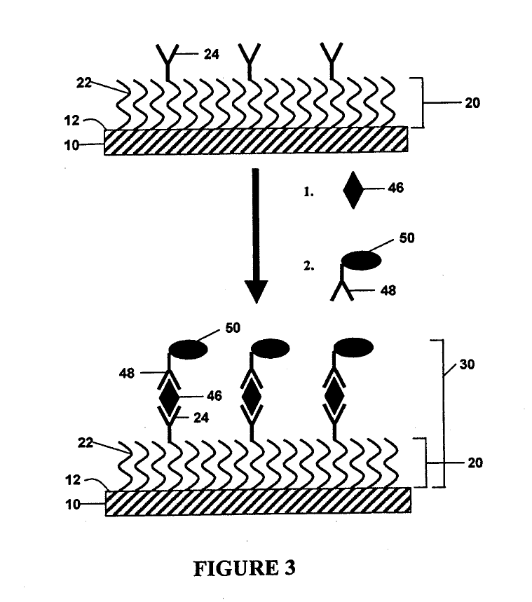

Once monolayer 20 is formed on the surface 12, and capture antibody 24 is immobilized, a sample may be contacted with the electrode for capture and detection of a target protein 46. As illustrated in FIG. 3, an analyte layer 30 is formed on the electrode 10, comprising functionalized monolayer 20 with immobilized capture antibody 24, a captured target protein 46, and a labelled detection antibody 48 directly or indirectly labelled with an enzyme label 50. Preferably, the detection antibody 48 is a monoclonal antibody.

In the depicted embodiment, capture antibody 24 is an antibody that recognizes target protein 46. Capture antibody 24 specifically binds the target protein 46, meaning with that it binds target protein 46 with greater affinity and selectivity than it binds other proteins that may be in solution with target protein 46.

Target protein 46 may be captured by contacting a sample containing target protein 46 with capture antibody 24 immobilized on functionalized monolayer 20.

The sample may be any sample in which it is desired to detect the presence of target protein 46, including a crude or partially purified cell lysate, a tissue culture medium containing secreted proteins, blood, serum, cerebrospinal fluid, saliva or urine. The contacting is performed under conditions that increase the specific binding between capture antibody 24 and target protein 46, for example at a temperature and for a duration, in the presence of any necessary cofactors.

A solution containing labelled detection antibody 48, labelled with enzyme 50 is added to the partially formed analyte layer 30, to bind labelled detection antibody 48 to captured target protein 46, and so as not to interfere with the interaction between capture antibody 24 and target protein 46. Binding of labelled detection antibody 48 to captured target protein 46 brings enzyme 50 into the analyte layer 30 in a manner that is dependent on the concentration of captured target protein 46.

Labelled detection antibody 48 is labelled either directly or indirectly with enzyme 50 prior to addition to the present method. To directly label detection antibody 48, protein cross-linking methods may be used, as described above. Alternatively, detection antibody 48 or enzyme 50 may be modified, for example by post-translational modification, with a chemical group at a specific site in the detection antibody 48 or enzyme 50, the chemical group being reactive with a reactive group in the other of detection antibody 48 or enzyme 50.

To indirectly label detection antibody 48 with enzyme 50, detection antibody 48 may be directly labelled with a ligand or affinity binding molecule, as described above for direct labelling with enzyme 50. The ligand or affinity binding molecule is any molecule that interacts with another molecule through a specific interaction, such as either half of a receptor/ligand pair which bind to each other through a specific, non-covalent affinity interaction. For example, the affinity binding molecule may be biotin, streptavidin, avidin, an ATP analogue, an ATP binding domain, imidazole, digoxigenin or a 6-histidine peptide. A molecule that recognizes the affinity binding molecule may then be attached to enzyme 50, so as to label detection antibody 48 with enzyme 50 through the interaction with the affinity binding group. Where such an affinity binding molecule, or the molecule that recognizes it, is a protein, recombinant cloning techniques may be used to express an enzyme as fusion protein with the molecule that recognizes the affinity binding molecule fused at one end of the enzyme. Standard recombinant cloning and expression methods are known in the art, and are described in standard molecular biology manuals and texts such as Sambrook et al. in Molecular Cloning: A Laboratory Manual, 3^{rd} Edition, Cold Spring Harbour, Laboratory Press. Alternatively, a spacer molecule can be used. A spacer molecule is a molecule that binds to the ligand or affinity binding molecule attached to detection antibody 48 for example through a complementary affinity binding molecule attached to the spacer molecule, and with a second ligand or affinity binding molecule attached to enzyme 50, through a second site on the spacer molecule. For example, detection antibody 48 and enzyme 50 may both be conjugated to a biotin molecule, and avidin or streptavidin is then used as the spacer molecule.

Enzyme 50 is an enzyme that catalyzes a reduction or an oxidation reaction, thus mediating electron transfer. The electron transfer may be between two substrates, or it may be between a substrate and a coenzyme, for example an oxidizing agent such as NAD⁺. The catalysis of electron transfer by enzyme 50 results in a change of oxidation state of the catalytic active site of enzyme 50 or of a coenzyme located at or in proximity to the catalytic active site. Thus, upon catalyzing a single reduction or oxidation reaction, enzyme 50 needs to be reset with respect to oxidation state in order to be ready to engage in a subsequent catalytic reaction, by interaction with an oxidizing or reducing agent.

Enzyme 50 may be, for example, an oxidoreductase, glucose oxidase, horse radish peroxidase, glucose-6-phosphate-dehydrogenase, catalase, peroxidase, microperoxidase, alkaline phosphatase, β-galactosidase, urease, β-lactamase, lactate oxidase or laccase.

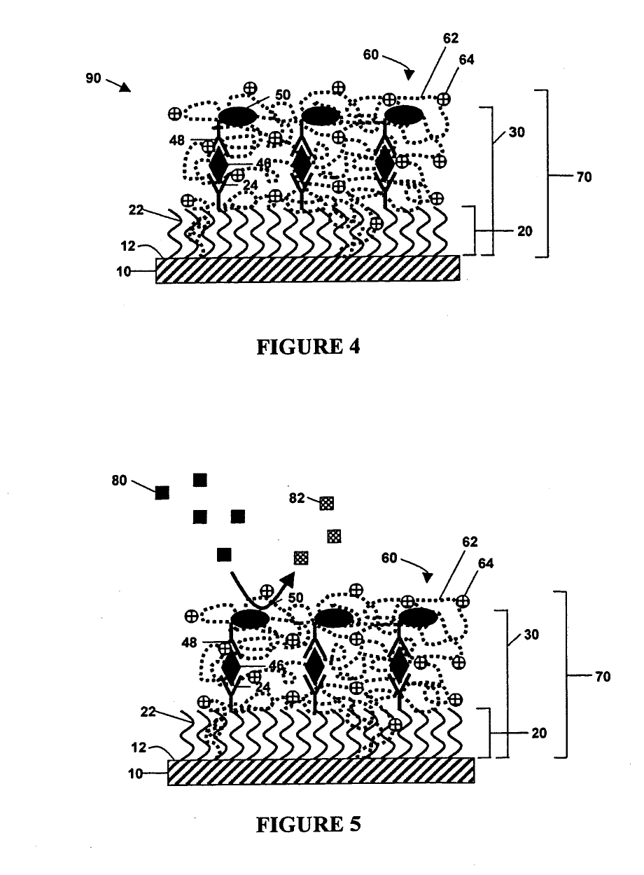

Analyte layer 30 is then layered with redox polymer 60 to form a bilayer 70, as shown in the schematic representation of the immobilized bilayer 70 in FIG. 4.

Redox polymer 60 comprises a polymer 62 complexed with a redox centre 64. The polymer 62 may be a conducting polymer, which is any polymer capable of conducting an electron flow, including polyvinylpyridine, polysiloxane, polypyrrole, polyquinone or polyvinylpyridine-co-acrylamide. For example, polymer 62 may be a conjugated polymer having a system of overlapping pi bonds along its backbone, as is the case in polyvinylpyridine. Alternatively, the redox polymer 60 may conduct electron flow by electron transfer between adjacent redox centres 64 that are complexed along the length of polymer 62 as described below.

Redox polymer 60 has a functional group that is complementary to that at the surface of functionalized monolayer 20. For example, in one embodiment, the functional group is a charged group that forms an electrostatic interaction with an opposite charge at the surface of monolayer 20. The charge may be associated with redox centre 64, which may be, for example, a metal cation. Alternatively, the charge may be associated with a functional group or substituent on the conductive polymer 62, for example, an ammonio group or a carboxylic group. Since the charge on redox polymer 60 is opposite to that of functionalized monolayer, the layering of redox polymer 60 onto the functionalized monolayer 20 occurs via an electrostatic interaction. There may be further electrostatic interactions between redox polymer 60 and the various protein components of the analyte layer 30, and any such interactions may assist in formation and/or stabilization of the bilayer 70 by maximizing the loading of redox polymer 60.

Redox centre 64 is a molecule, ion or complex, including a chelated metal cation complex, that exhibits a reversible electrochemistry and which is coordinated by polymer 62. Redox centre 64 therefore can cycle between oxidized and reduced states upon contacting with a suitable electron acceptor or donor. In certain embodiments, redox centre 64 may be quinone, ferrocene, osmium (4,4′-dimethyl-2,2′-bipyridine)₂ or tetrathiafulvalene, or may be Ru, Co, Fe or Rh complexes. Redox centre 64 is chosen so that its redox potential is in a similar range as the enzyme catalytic active site, or coenzyme of enzyme 50, to allow for electron transfer to occur.

Polymer 62 is complexed at positions along its length with redox centre 64, for example by a covalent bond, an electrostatic interaction, or through forming a coordination bond with a chelated ion complex, to form redox polymer 60. The redox polymer 60 is capable of undergoing redox cycling reactions at each redox centre 64, which cycling can be measured through electrically contacting polymer 62 with electrode 10 and measuring current flow, as will be apparent to a skilled person.

To coat the analyte layer 30 with redox polymer 60, a solution containing redox polymer 60 is contacted with analyte layer 30, resulting in a stable bilayer which is immobilized at electrode surface 12 through interaction of the complementary functional groups. Redox polymer 60 will coat the complex of enzyme 50, labelled detection antibody 48, captured target protein 46 and capture antibody 24, coming into contact with functionalized monolayer 20, and penetrating functionalized monolayer 20 in places to contact electrode surface 12 where defects exist in the monolayer. Since electrons can be transferred across the monolayer, either by conductance or via tunnelling, in this way, redox polymer forms an electrical connection between the catalytic active site of enzyme 50 and electrode 10, allowing for the measurement of electron transfer catalyzed by enzyme 50.

To detect the presence of capture target protein 46, as shown in FIG. 5, a substrate 80 for enzyme 50 is added to the bilayer 70 immobilized on electrode surface 12 in a buffer and under conditions suitable for enzyme 50 to catalyse oxidation or reduction of the substrate 80. As stated above, substrate 80 can be either oxidized or reduced to form product 82 in a reaction catalyzed by enzyme 50. Thus, the substrate 80 possesses a first oxidation state, and is converted by enzyme 50 to product 82 having a second oxidation state.

The specific identity of substrate 80 will depend on which enzyme 50 is used. For example, the substrate may be glucose, hydrogen peroxide, glucose-6-phosphate, phenylphosphate, p-aminophenylphosphate, p-aminophenyl-β-galactoside, urea or benzyl penicillin.

Upon addition of substrate 80, the enzyme-catalyzed oxidation or reduction reaction is carried out in the bilayer by enzyme 50 to yield product 82. Upon catalysing an electron transfer from or to the substrate 80, the catalytic active site of enzyme 50, or a coenzyme of enzyme 50 then undergoes electron transfer with the redox centres 64 in redox polymer 60, which together with monolayer 20 will form a conductive path between the electrode 10 and the catalytic active site or coenzyme through the polymer 62. Similarly, the polymer 62 is in contact with monolayer 20, which can transfer electrons to and from the electrode surface 12, either through conductance or tunneling of the electrons, thus allowing for the electrode 10 to recycle the oxidation state of the redox centre 64. The electron transfer that occurs between redox centre 64 and enzyme 50 can thus be detected indirectly using amperometric measurement.

Briefly, the starting redox state of redox centres 64 in redox polymer 60 are such that no electrons can transfer between the redox polymer 60 and electrode 10, even upon application of a potential difference by biasing source 8. For example, if electrode 10 is a cathode, and redox centres 64 in redox polymer 60 are to be reduced, the redox centres 64 are initially in a reduced state, meaning that no current will flow at electrode 10. Current flow is dependent upon electron transfer from the active site of enzyme 50 or from a coenzyme of enzyme 50 to an adjacent redox centre 64 in redox polymer 60. Enzyme 50 will catalyze the reduction of substrate 80, thereby becoming oxidized. Enzyme 50, or a coenzyme of enzyme 50, will be reset to a reduced state by accepting an electron from an adjacent redox centre 64. The redox centre will thus be oxidized, and will accept an electron from the next adjacent redox centre 64, which will in turn be oxidized as the first redox centre 64 is reduced, and so on, until the last redox centre immediately adjacent to the monolayer is oxidized and then subsequently becomes reduced by accepting an electron from electrode 10, which is transferred across monolayer 20, resulting in current flow at electrode 10.

As mentioned above, the reference electrode 6 will also be in contact with the solution 7 containing substrate 80 and which is contacting electrode 10, and the solution will contain electrolytes which will thus complete a circuit containing electrode 10, allowing for current to flow through electrode 10. Suitable electrodes that can be used as reference electrode 6 are known, for example, an Ag/AgCl electrode can be used.

A potential difference is applied between electrodes 6 and 10 in order to catalyze the electron transfer reactions at the respective electrodes.

Thus, detection of current flow between substrate 80 in solution 7 and the electrode 10, by way of the catalytic active site of the enzyme, the redox centres of the polymer and the contact of the polymer with the electrode, allows for the amperometric measurement of the concentration of captured target protein 46. Biasing source 8 applies a constant potential between reference electrode 6 and electrode 10, while substrate 80 is in solution 7. The applied potential is chosen to be such as to drive an electron transfer between the enzyme 50 and redox polymer 60. Typically, the applied potential is at least 50 mV more positive than the redox potential of an oxidative reaction of the redox centres 64 of redox polymer 60, or at least 50 mV more negative than a reductive reaction of the redox centres 64, depending on whether electrode 10 is acting as an anode or cathode.

The current generated as a result of electron transfer catalysed by enzyme 50 will be directly proportional to the concentration of enzyme 50, and therefore to the concentration of captured target protein 46, allowing for quantification of the concentration of target protein 46. Since surface 12 is protected by monolayer 20, inhibiting non-specific binding of the various protein reagents, and thus reducing the amount of non-specifically bound enzyme 50, the current that flows at electrode 10 should be that which is derived from molecules of enzyme 50 that are specifically associated with captured target protein 46. A skilled person will understand how to perform a standard curve with known concentrations of target protein 46 so as to correlate the level of detected current with detection of a given concentration of protein.

As will be appreciated by a skilled person, surface 12 of electrode 10 is rinsed between steps of forming the bilayer 70 in the present method and prior to detecting the presence of target protein 46, in order to eliminate excess unreacted reagents. For example, surface 12 may be rinsed with blank buffer to remove any unbound capture antibody 24 and unreacted cross-linker after immobilization of capture antibody 24 on functionalized monolayer 20.

Optionally, regions of surface 10 which are not covered by protective monolayer 20 may be blocked with an inert blocking agent that will not participate in the redox reactions, is not electrically active, and which will have minimal affinity for any of the reaction components. One such suitable blocking agent is bovine serum albumin protein.

For each of the above steps, the appropriate solution is added to the surface 12 of electrode 10 using a liquid cell, which may be a flow cell, as is known in the art, or by pipetting directly onto surface 12, either manually or using an automated system. The liquid cell can form either a flow through liquid cell or a stand still liquid cell.

While the above embodiments describe using a capture antibody to capture a target protein, the molecule used to capture the target molecule may be any capture molecule that can be immobilized at the functionalized surface of the monolayer, and which can specifically bind the target molecule. For example, the capture molecule may be a protein, an antibody including a monoclonal antibody, an antibody fragment, a receptor, a receptor fragment, a ligand, an inhibitor, a small molecule, a nucleic acid, a hormone or a non-cleavable substrate analogue.

The target molecule may be any molecule which is desired to be detected and quantified in a sample, and which can be captured from the sample using a capture molecule. Thus, the target molecule may be, for example, a protein, a peptide, a receptor, a receptor fragment, a nucleic acid, a ligand, an inhibitor, a small molecule, a hormone or a non-cleavable substrate analogue.

Similarly, the detection molecule has been described above as an antibody. Although an antibody is the preferred detection molecule, since it allows for a specific and sensitive method of detection. However, it will be appreciated that any molecule that specifically binds to captured target molecule could be used for detection, provided that the detection molecule can be labelled directly or indirectly with the enzyme that is to be used to catalyze electron transfer.

As noted, in electrochemical assays immobilization of protein reagents at an electrode typically requires a chemical reaction directly between the electrode and the protein that is to be immobilized. Such immobilization methods can result in random orientation of the protein on the electrode surface, providing sub-optimally arranged protein for capturing of a target molecule from solution, or alternatively, denaturation of the protein, which can also result in non-specific binding of other protein reagents that are in solution. Furthermore, proteins typically are large, bulky molecules and tend not to conduct or tunnel electrons very efficiently.

Proteins can be advantageously immobilized on the monolayer in the present method due to the monolayer which provides a functionalized surface at which proteins can readily be immobilized in a controlled manner at a specific site in the protein and at a concentration at which capture of a target molecule is optimized.

The protective monolayer 20 increases the specificity and sensitivity of the amperometric assay, particularly where protein reagents are to be used or target proteins are to be detected. The inclusion of the monolayer 20 protects the electrode surface, preventing the non-specific adherence of reagents, particularly protein reagents such as a protein used as a capture molecule and the enzyme used to catalyze the oxidation or reduction of the substrate, to the bare surface of the electrode and as a result, reducing non-specific background signal and allowing for lower amounts of target molecule to be detected. Furthermore, the monolayer can be specifically functionalized to allow for control of the orientation and spacing of immobilization of the capture molecule. This helps to stabilize protein reagents, for example by minimizing denaturation of a protein capture molecule, and allows for optimization of conditions for capturing the target molecule from the sample solution. The functionalized monolayer also assists in forming the bilayer system by interaction with complementary functional groups on the redox polymer.

Conveniently, labelling of the test sample or cross-linking of the detection antibody or enzyme is not required. Moreover, the present assay has a broad linear detection range with a low detection limit. Where the target molecule is a protein, the present method allows for detection of as little as 2 fg of analyte protein in a test sample, at concentrations as low as 2 pg/mL.

Due to electrode technology that allows for miniaturization of electrodes, the above method can be designed to be carried out in small volumes, for example, in as little as 1 μl volumes. In combination with the very low detection limit, this makes the present method a highly sensitive method of detecting protein in a sample, which is applicable for use in point-of-care and in-field applications, including disease diagnosis and treatment, environmental monitoring, forensic applications and molecular biological research applications including proteomic approaches.

The above-described embodiment is a sandwich-type assay in which the target molecule is immobilized and detected by a sandwich of the capture molecule and the detection molecule. Within the dynamic range of the assay, the measured current is directly proportional to the concentration of captured target molecule. While such an assay format is typically preferred due to a higher sensitivity and the direct correlation between protein concentration and measured current, it will be appreciated that the present method using a protective monolayer at the electrode surface, and the formation of the bilayer prior to detection can be performed as a competitive assay. In such case, the detection molecule is a competitor of the target molecule, for example, a purified protein that is the same as the target molecule, or a protein fragment of the same protein, which binds to the capture molecule in a competitive manner with the sample-derived target molecule. The capture step in which the sample-derived target molecule is immobilized thus prevents the detection molecule, the competitor protein, from being immobilized in the analyte layer, thereby preventing the enzyme from being incorporated into the analyte layer. In this way, the greater the amount of target molecule in the sample, the lower the concentration of enzyme that is incorporated into the bilayer, and the lower the current will be. The current flow that is measured is indirectly proportional to the amount of target molecule in the sample.

A device 90, as depicted in FIG. 4, is also contemplated. Device 90 comprises electrode 10 having surface 12, on which functionalized monolayer 20 is located. Capture antibody 24 is immobilized on the surface of monolayer 20. In the depicted embodiment, capture antibody 24 binds target protein 46, which in turn binds to detection antibody 48, labelled with enzyme 50, all of which forms an analyte layer 30. The analyte layer 30 is layered with redox polymer 60 to form bilayer 70. Redox polymer 60 is held in place through interactions between the end functional groups at the surface of monolayer 20 and complementary functional groups located in redox polymer 60.

Since the analyte layer 30 contains the target protein 46 that is to be detected, it is not possible to preform the bilayer 70 or even the analyte layer 30 on the electrode surface 12 in advance of obtaining a particular sample to be tested. However, as a skilled person will appreciate, the electrode surface 12 may be modified to have the functionalized monolayer 20 and immobilized capture antibody 24, and stored until obtaining the protein sample to be tested.

In an alternate embodiment of device 90, if a competitive assay is to be performed, the device 90 comprises the target protein 46 and the detection antibody 48, which is a competitor of target protein 46 and which is labelled with enzyme 50, both bound to different distinct molecules of capture antibody 24 within analyte layer 30.

The present methods and devices are well suited for high throughput processing and easy handling of a large number of protein samples. To assist in high volume processing of samples, the present devices may be adapted for use in an array of electrodes. Multiple devices 90 may be formed in an array, for use in high throughput detection methods as described above. Each of device 90 in the array may comprise a different capture antibody 24, for detecting a number of different proteins simultaneously. Alternatively, each device 90 in the array may comprise the same capture antibody 24, for use in screening a number of different samples for the same protein.

Alternatively, multiple electrodes 10 can be arranged into an array, partially prepared as described above, for use with protein samples once acquired.

Each electrode 10 may be located within a discrete compartment, for ease of applying the same or different target protein 46, labelled detection antibody 48 and enzyme 50, followed by redox polymer 60, to each surface 12 of each electrode 10. Alternatively, each electrode 10 can be arrayed so as to contact a single bulk solution. An automated system can be used to apply and remove fluids and sample to each electrode 10.

A different capture antibody 24 for detecting a particular protein within a sample may be immobilized on the functionalized monolayer 20 on respective electrodes 10. Each electrode 10 may then be contacted with the same sample so as to detect multiple proteins within a single sample at one time.

Alternatively, multiple electrodes 10 may be arranged in an array such that each individual electrode has the same capture antibody 24 immobilized on functionalized monolayer 20. A different sample may then be contacted with each respective electrode 10. In this way a large number of samples may be screened for a particular protein.

Kits or commercial packages for carrying out the described method are also contemplated. The kit or commercial package contains an electrode 10 having a partially formed analyte layer 30 of functionalized monolayer 20 and immobilized capture antibody 24 or an array of such electrodes, a redox polymer 60 for layering on the completely formed analyte layer and instructions for detecting a protein in a sample using the above described method. The kit or commercial package may also include a detection antibody 48 and may further include enzyme 50, which may be conjugated as a labelled detection antibody-enzyme conjugate, or which may be included with instructions for labelling detection antibody 48 with enzyme 50. The kit or commercial package may further include substrate for the enzyme 50.

All documents referred to herein are fully incorporated by reference.

### EXAMPLES

The above method was performed to detect the venom protein β-BuTx from Bungarus multicinctus using a charged protective monolayer of mercaptoundecanoic acid self-assembled on a gold electrode via thiol chemistry. The capture molecule used was a monoclonal antibody directed towards venom from Bungarus multicinctus, which was crosslinked to the free carboxyl group of the monolayer using EDC/NHS crosslinking methods. The remaining electrode surface not covered by analyte layer was blocked using bovine serum albumin. The venom protein, in either PBS or in serum, was captured and then recognized with a second anti-Bungarus multicinctus venom antibody conjugated to biotin. Avidin-conjugated horseradish peroxidase (“A-HRP”) was added to complete the formation of the analyte layer. Polyvinylpyridine-co-acrylamide complexed with Os(4,4′-dimethyl-2,2′-bipyridine)₂Cl^{+/2+} was added as redox polymer, and current was measured in a solution of 5 mM hydrogen peroxide. As little as 2 fg and 10 fg of protein was detected in PBS and serum, respectively.

### EXPERIMENTAL

Materials and Apparatus: unless otherwise stated, chemicals were obtained from Sigma-Aldrich (St Louis, Mo.) and used without further purification. The redox polymers used in this study were poly(vinylimidazole-co-acrylamide) (PVIA-Os); poly(vinylimidazole-co-acrylamido-2-methyl-1-propanesulfonic acid) (PVIAMP-Os); poly(vinylimidazole-co-acrylic acid) (PVIAA-Os); poly(vinylpyridine-co-acrylamide) (PVPA-Os); and poly(vinylpyrridine-co-acrylic acid) (PVPAA-Os). Synthesis of the redox polymers was described elsewhere (Gao et al. (2002) Agnew Chem, Int. Ed. 41: 810-813; Campbell et al. (2002) Anal. Chem. 74: 158-162). To demonstrate the “proof of concept”, β-BuTx was selected as the model analyte since rabbit polyclonal antibody and monoclonal antibodies (mAb5, mAb11 and mAb15) to this toxin were previously produced and available (Selvanayagam et al. (2002) Biosens Bioelectron. September 17: 821-826). β-BuTx (molecular weight ˜8 KDa) was purchased from Sigma (St. Louis, Mo., catalogue number T5644). Among the three monoclonal antibodies, mAb15 showed the strongest bioaffinity towards β-BuTx and was therefore selected for this study. The biotinylation of mAb15 was performed as described previously (Le et al. (2002) J Immunol Methods. 260: 125-136). A-HRP was obtained form Sigma.

Electrochemical experiments were carried out using a CH Instruments model 660A electrochemical workstation coupled with a low current module (CH Instruments, Austin, Tex.). The three-electrode system consisted of a 2-mm-diameter gold working electrode, a miniature Ag/AgCl reference electrode. Phosphate-buffered saline (PBS, pH 7.4), consisting of 0.15 M NaCl and 20 mM phosphate buffer, was used as the supporting electrolyte.

Protein array fabrication: to fabricate the sensor array, a titanium adhesion layer of 25-50 Å was electron-beam evaporated onto a silicon wafer followed by 2500-3000 Å of gold. Before antibody modification, the gold coated wafer was thoroughly cleaned with freshly prepared piranha solution (98% H₂SO₄/30% H₂O₂=3/1) and rinsed with Milli-Q water followed by 10 min in ultrasonic bath in absolute ethanol. (Caution—piranha solution is a powerful oxidizing agent and reacts violently with organic compounds.) The gold surface was then modified immediately after the cleaning step. Initial thiol adsorption was accomplished by immersing the gold substrate in 10 mM MUA (mercapto-undecanoic acid) in absolute ethanol overnight at room temperature. MUA solutions were freshly prepared before each experiment. The electrodes were rinsed with Milli-Q water and activated with 100 mM of 1-ethyl-3(3-dimethyl-aminopropyl)-carbodiimide (EDC) and 40 mM of N-hydroxysulfosuccinimide (NHS) in water. A patterned 1-mm thick adhesive spacing/insulating layer with a screen-printed Ag/AgCl layer and a hydrophobic layer were assembled on the top of the slide. The diameter of the individual sensor was 2.0 mm and that of the top hydrophobic pattern was 4 mm. Protein A-purified rabbit IgG anti-β-BuTx antibody (0.10 mg/mL in PBS) was applied on each of the individual sensors and incubated for 3 h at room temperature. After rinsing with washing buffer (PBS, containing 0.050% Tween-20 (PBS-T)), the unoccupied sites were blocked by incubating with 1.0% bovine serum albumin (BSA) in PBS containing 0.50% Tween 20 overnight at 4° C. The array was rinsed with washing buffer then stored at 4° C. in PBS solution until used.

Protein detection: β-BuTx incubation and its electrochemical detection were carried out as follows. The electrode was placed in a moisture-saturated environmental chamber. Aliquots of β-BuTx solution (2.0 μL) were placed on the sensor and incubated for 30 min. After washing for 10 min in a vigorously stirred PBS solution and drying, biotinylated mAb15 (5.0 μL) was added and the chip was incubated for 30 min. After another washing and drying cycle, A-HRP (5.0 μL) was dispensed onto each chip and incubated for 10 min. The chip was washed, dried and the redox polymer (10 μL) was applied onto the electrode and incubated for 10 min. Electrochemical characterization was carried out with a gold electrode. An Ag/AgCl electrode was used as the reference electrode and a platinum wire as the counter electrode. Detection of β-BuTx was performed on the protein array. The individual sensor remained open-circuit until a 10 μl aliquot of PBS test solution was applied. Withdrawal of the test solution effectively disabled the sensor. The catalytic response was evaluated by amperometry at a constant potential (0.15 V) in PBS containing 5.0 mM H₂O₂. Where low toxin concentrations were used, smoothing was applied after each measurement to remove random noise. All incubations and measurement were performed at room temperature. All potentials reported in this work were referred to the Ag/AgCl reference electrode.

### RESULTS AND DISCUSSION

In a previous method involving nucleic acids as the capture, target and detection molecules, the thiol-containing capture nucleic acid was directly immobilized on a gold electrode, and the monolayer was formed by assembling monolayer component molecule around the DNA (Xie et al., Anal. Chem. (2004) 76: 1611-1617; Xie et al., Nucl. Ac. Res. (2004) 32(2): e15). The present method of chemical coupling of the capture molecule to the surface of the monolayer, instead of direct adsorption onto the bare electrode surface, has three distinct advantages: (i) it provides improved stability of the immobilized capture molecules which are proteins, (ii) it inhibits the non-specific adsorption of protein reagents onto the bare gold electrode, and (iii) the surface coverage of the capture molecule can be manipulated to optimize conditions for target molecule binding, particularly where the target molecule is a protein.

Formation of electroactivated bilayer: the fabrication of the protein array for use in EEIA requires a series of surface chemical reactions. These steps are as follows: (1) formation of a self-assembled monolayer of MUA, (2) reaction of the MUA monolayer with EDC-NHS, (3) covalent attachment of antibody onto the array and (4) treatment of the unreacted sites on the electrode surface with a blocking agent, BSA. The fabrication of the array was monitored by different methods such as optical ellipsometric, contact angle, surface coverage and QCM measurements. In step 1, a monolayer of MUA is self-assembled onto the gold substrate. Similar to those reported in earlier methods, all data obtained indicated a single molecular layer of MUA coated on the gold electrode (Finklea, H. O. in Electroanalytical Chemistry, Bard A. J. and Rubenstein, I., eds., Marcel Dekker: New York, 1996, Vol. 19: 109-335; Ulman, A., An Introduction to Ultrathin Organic Films from Langmuir Blodgett to Self-Assembly, Academic Press: San Diego, Calif., 1991). In step 3, antibody is covalently attached to the surface of the MUA monolayer. In the final step of the electrode fabrication, the portions of the electrode not protected by the monolayer were blocked by reacting with BSA, resulting in a surface that is resistant to non-specific adsorption of proteins. This is advantageous significant non-specific adsorption of proteins, particularly A-HRP conjugates, would undoubtedly compromise the accuracy of the monitoring of the protein binding events. BSA is well known for its ability to resist the non-specific adsorption of proteins (Steinitz, (2002) M. Anal. Biochem. 282: 232-238), and BSA blocked surfaces are currently used in many protein assays.

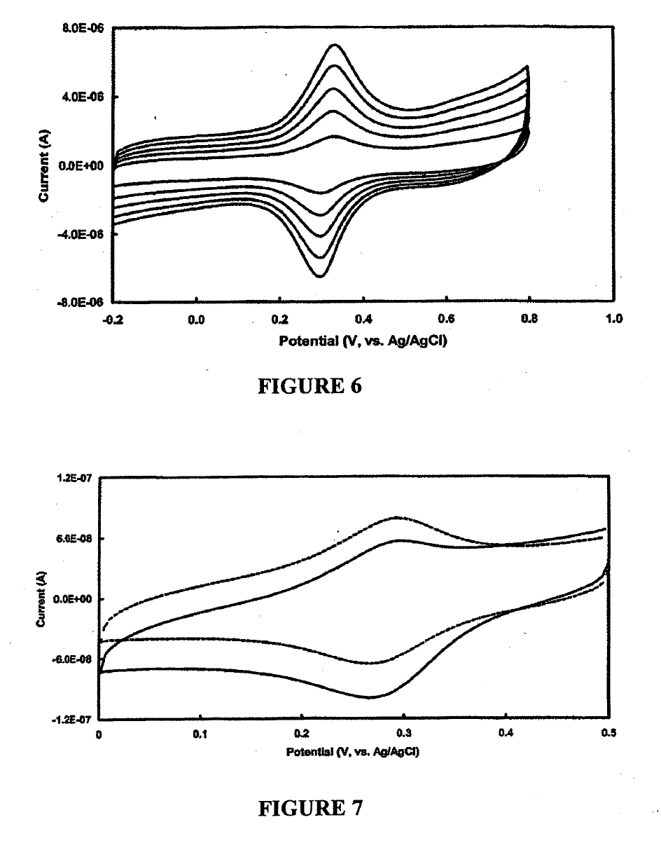

PVIA-Os, PVIAMP-Os, PVIAA-Os, PVPA-Os, and PVPAA-Os were first tested for their ability to form stable bilayers. It was found that among these redox polymers, PVPA-Os is the best in terms of stability of the bilayer and the amount of redox polymer being immobilized on the biosensor surface. This is likely due to the partial protonation of acrylamide moieties at pH 7.4, increasing the net positive charge of the redox polymer, thereby reinforcing the formation of the bilayer, which brings the osmium redox centres in the proximity of A-HRP. Therefore, PVPA-Os was used throughout. As expected, the MUA-antibody monolayer alone impedes electron transfer between the gold electrode and the solution species. No detectable current was observed when tested by cyclic voltammetry in a 0.50 M Na₂SO₄ solution containing 2.5 mM ferricyanide. However, since the redox polymer is positively charged and the electrode is negatively charged, a brief soaking of the electrode in the 5.0 mg/mL PVPA-Os solution resulted in the formation of an analyte/redox polymer bilayer on the electrode via the layer-by-layer electrostatic self-assembly (Decher, G. (1997) Science 277: 1231-1237). As illustrated in FIG. 6 (scanned at 100, 200, 300, 400 and 500 mV/s, innermost curve to outermost curve, respectively), the redox polymer coated electrodes performed as expected for a reversible surface immobilized redox couple (Bard, A. J. and Faulkner, L. R. Electrochemical Methods, John Wiley & Sons: New York, 2001, p. 590). The peak currents were found to be linear with potential scan rate up to 500 mV/s and the ratio of the anodic to the cathodic charge obtained by integrating the current peaks at a very slow scan rates was very close to unit, showing that the charge transfer and counter-ion transfer within the film and the charge transfer from the redox polymer film to the electrode are rapid. A derivation from linearity accompanied by an observable tailing current, occurred when increasing potential scan rate beyond 1.0 V/s. The voltammograms were almost symmetrical at low potential scan rates and the peak-to-peak potential separation (ΔEp) was less than 20 mV. Little change after exhaustive washing with water and PBS and after numerous repetitive potential cycling between −0.2 V and +0.8 V, revealing a highly stable surface immobilized electrostatic bilayer on gold electrode. The presence of HRP in the bilayer did not appreciably alter the electrochemistry of the redox polymer. Later experiments in substrate solution showed that HRP in the bilayer retains its activity. Such results ascertain that the osmium redox centres are in electrical contact with the electrode surface and participate in reversible heterogeneous electron transfer. The total amount of bound osmium redox centres, 2.3-6.0×10⁻¹⁰ mole/cm², depending on the amount of β-BuTx bound to the electrode, was estimated from the area under either the oxidation or the reduction current peak corrected for the background current.

Feasibility of protein detection: in the first feasibility study, β-BuTx standard solutions were tested on the protein array. Upon application at room temperature to the monolayer with capture Ab on the electrode surface, β-BuTx in the solution was selectively bound by the capture Ab and immobilized in the vicinity of the electrode surface. Repeated rinsings with PBS were performed to remove excess β-BuTx. A-HRP was incorporated in the analyte layer via biotin-avidin interaction during subsequent incubation with the second antibody and A-HRP solution. Typical cyclic voltammograms of the sensor reacted with β-BuTx in PBS (dashed curve) and in a 2.0 mM H₂O₂ (solid curve) are shown in FIG. 7. Catalytic current was observed in the presence of H₂O₂ due to the presence of HRP in the bilayer. In a control experiment, BSA failed to capture any β-BuTx and therefore A-HRP was not incorporated in the analyte layer. Identical voltammograms were then obtained in PBS and PBS containing H₂O₂ (not shown). No catalytic currents in voltammetry were noticed.

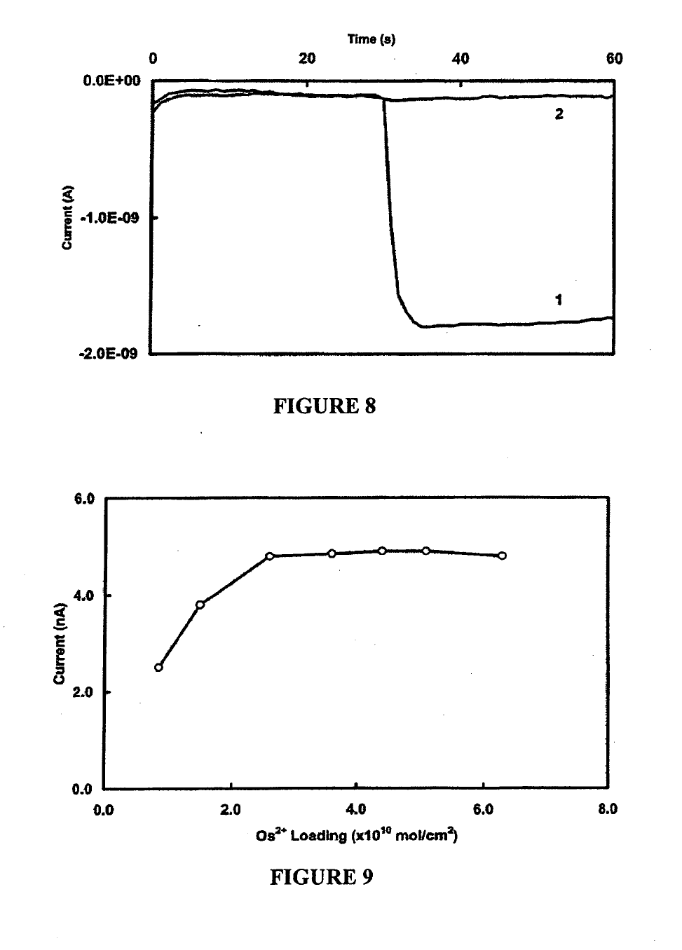

When the fabricated bilayer was immersed in PBS, the reduction current in amperometry increased by 1.8 nA at 0.15 V upon adding 2.0 mM H₂O₂ to PBS (FIG. 8, trace 1). In an identical experiment (control experiment) where BSA was immobilized on the monolayer surface, negligible change of current was observed (FIG. 8, trace 2). The amperometric results complemented the cyclic voltammetric data obtained earlier and confirmed that β-BuTx was successfully detected with high specificity. As expected, the amperometric signal is strongly dependent on the redox polymer loading. The oxidation current increased with increasing the amount of redox polymer up to 2.0×10⁻¹⁰ mole/cm² and then started to level off (FIG. 9). It was found that maximal loading of 4.0-6.0×10⁻¹⁰ mole/cm² could easily be achieved after 5-10 min of adsorption in the 5.0 mg/mL redox polymer solution. To safeguard the amperometric sensitivity, maximal loading was always used for protein detection.

The Os(bpy)²⁺ sites of the redox polymer overcoating effectively interact with the HRP incorporated in the bilayer and serves to reset the enzyme upon enzymatic reduction of H₂O₂. At the applied potential of 0.15 V, the thus oxidized redox polymer is subsequently reduced, forming a substrate-recycling mechanism in the bilayer, as described by the following equations:

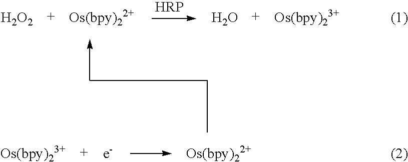

When the reduction potential for Os³⁺ is sufficient, the overall reaction rate and hence the sensitivity of the system is determined by equation (1), or in other words, by the apparent activity of HRP in the bilayer.

To test for possible catalysis by HRP through direct electron-exchange with the substrate electrode, a sensor without applying the redox polymer was fabricated and its voltammogram was measured in PBS containing H₂O₂. Comparison of the voltammetry and amperometry with that of an electrode that was not treated by β-BuTx showed no measurable difference. Furthermore, while H₂O₂ is catalytically electroreduced already at a potential as positive as 0.30 V vs Ag/AgCl on the electrode with the redox polymer overcoating, electroreduction of H₂O₂ was not observed on a gold electrode exposed to the HRP solution or PVPA-Os solution at potentials negative of 0.20 V, ruling out the possibility that the reduction of hydrogen peroxide is catalyzed by immobilized HRP or by PVPA-Os.

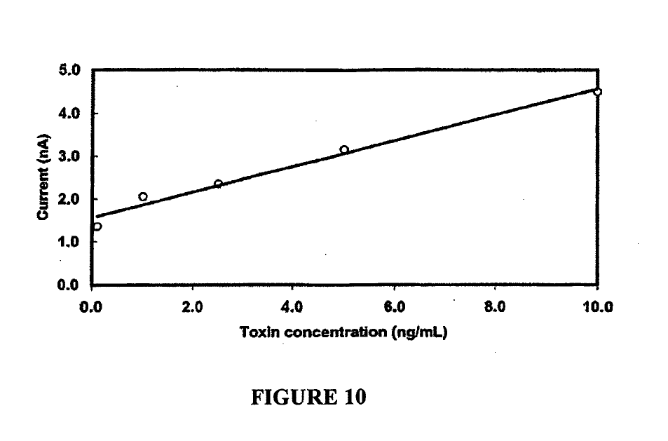

Calibration curve for β-BuTx: FIG. 10 shows representative amperometric data obtained from the protein array treated with solutions of increasing concentrations, from 10 pg/mL to 10 ng/mL. As the concentration of β-BuTx was increased, the H₂O₂ reduction current increased accordingly in amperometry. The toxin concentrations were proportional to the reduction currents indicating that the biosensor can be used for quantification purpose. Under optimal experimental conditions, a dynamic range was found to be from 2.0 pg/mL to 10 ng/mL with a detection limit of 1.0 pg/mL estimated based on 3-fold measurement of noise levels. It was found that a practically constant current (saturation current) was observed at a β-BuTx concentration of 50-100 ng/mL. Higher detection limit, 10.0 pg/mL, was observed when working with serum samples.

As can be understood by one skilled in the art, many modifications to the exemplary embodiments described herein are possible. The invention, rather, is intended to encompass all such modification within its scope, as defined by the claims.

What is claimed is:

1. A method of electrochemically detecting a target molecule in a sample, comprising:
coating an electrode with a monolayer capable of immobilizing a capture molecule thereon and of transferring electrons there across, thereby inhibiting non-specific binding of protein molecules at the electrode;
immobilizing the capture molecule on the monolayer;
adding a sample containing a target molecule to be captured by the capture molecule;
adding a detection molecule that specifically binds one of the capture molecule or the target molecule, wherein the detection molecule is labelled with an enzyme capable of oxidizing or reducing a substrate;
adding a redox polymer that interacts with the monolayer and that together with the monolayer forms a conductive path from the enzyme to the electrode;
adding the substrate to be oxidized or reduced by the enzyme; and
detecting current flow at the electrode.

2. The method of claim 1, wherein the detection molecule specifically binds to the target molecule and the detection molecule comprises an antibody.

3. The method of claim 1, wherein the detection molecule specifically binds to the capture molecule and the detection molecule comprises a competitor of the target molecule.

4. The method of claim 1 wherein the redox polymer has a charged group which interacts electrostatically with the monolayer through an oppositely charged group on the monolayer.

5. The method of claim 4 further comprising rinsing the electrode after each of said coating, said immobilizing, said adding the sample, said adding the detection molecule and said adding the redox polymer.

6. The method of claim 5 wherein the sample is a crude cell lysate, a partially purified cell lysate, a tissue culture medium containing secreted proteins, blood, serum, cerebrospinal fluid, saliva or urine.

7. The method of claim 6 wherein the capture molecule comprises a protein, an antibody, a monoclonal antibody, an antibody fragment, a receptor, a receptor fragment, a ligand, an inhibitor, a small molecule, a nucleic acid, a hormone or a non-cleavable substrate analogue.

8. The method of claim 7 wherein the target molecule comprises a protein, a peptide, a receptor, a receptor fragment, a nucleic acid, a ligand, an inhibitor, a small molecule, a hormone or a non-cleavable substrate analogue.

9. The method of claim 8 wherein the monolayer comprises mercaptoundecanoic acid or mercaptohexadecanoic acid.

10. The method of claim 9 wherein in the enzyme comprises an oxidoreductase, glucose oxidase, horse radish peroxidase, glucose-6-phosphate-dehydrogenase, catalase, peroxidase, microperoxidase, alkaline phosphatase, β-galactosidase, urease, β-lactamase, lactate oxidase or laccase.

11. The method of claim 10 wherein the substrate comprises glucose, hydrogen peroxide, glucose-6-phosphate, phenylphosphate, p-aminophenylphosphate, p-aminophenyl-β-galactoside, urea or benzyl penicillin.

12. The method of claim 11 wherein the electrode comprises carbon, a metal, a metal oxide or a conductive polymeric material.

13. The method of claim 12 wherein the electrode comprises carbon paste, carbon fiber, graphite, glassy carbon, gold, silver, copper, platinum, palladium, indium tin oxide, poly(3,4-ethylenedioxythiophene) (PEDOT) or polyaniline.

14. The method of claim 13 wherein the redox polymer comprises quinone, ferrocene, osmium (4,4′-dimethyl-2,2′-bipyridine)₂, tetrathiafulvalene, an Ru complex, a Co complex, an Fe complex, or an Rh complex as a redox centre.

15. The method of claim 14 wherein the redox polymer comprises polyvinylpyridine, polysiloxane, polypyrrole, polyquinone or polyvinylpyridine-co-acrylamide.

16. A method of providing an electrochemical cell for detecting a target molecule in a sample, comprising:
coating an electrode with a monolayer capable of immobilizing a capture molecule thereon and of transferring electrons there across, thereby inhibiting non-specific binding of protein molecules at the electrode; and
immobilizing the capture molecule thereon.

17. A device for performing an electrochemical assay, comprising:
a monolayer formed on a surface of an electrode, said monolayer capable of immobilizing a capture molecule thereon and of transferring electrons there across, thereby inhibiting non-specific binding of protein molecules at the electrode; and
a capture molecule immobilized on the monolayer.

18. The device of claim 17 further comprising:
a target molecule captured by the capture molecule;
a detection molecule specifically bound to one of the capture molecule or the target molecule, wherein the detection molecule is labelled with an enzyme capable of oxidizing or reducing a substrate; and
a redox polymer forming an interaction with the monolayer and that together with the monolayer forms a conductive path from the enzyme to the electrode.

19. The device of claim 18 wherein the detection molecule is specifically bound by the target molecule and the detection molecule comprises an antibody.

20. The device of claim 18 wherein the detection molecule is specifically bound by the capture molecule and the detection molecule comprises a competitor of the target molecule.

21. The device of claim 18 wherein the interaction formed between the redox polymer and the monolayer is an electrostatic interaction.

22. The device of claim 21 wherein the capture molecule comprises a protein, an antibody, a monoclonal antibody, an antibody fragment, a receptor, a receptor fragment, a ligand, an inhibitor, a small molecule, a nucleic acid, a hormone or a non-cleavable substrate analogue.

23. The device of claim 22 wherein the target molecule comprises a protein, a peptide, a receptor, a receptor fragment, a nucleic acid, a ligand, an inhibitor, a small molecule, a hormone or a non-cleavable substrate analogue.

24. The device of claim 23 wherein the monolayer comprises mercaptoundecanoic acid or mercaptohexadecanoic acid.

25. The device of claim 24 wherein in the enzyme comprises an oxidoreductase, glucose oxidase, horse radish peroxidase, glucose-6-phosphate-dehydrogenase, catalase, peroxidase, microperoxidase, alkaline phosphatase, β-galactosidase, urease, β-lactamase, lactate oxidase or laccase.

26. The device of claim 25 wherein the substrate comprises glucose, hydrogen peroxide, glucose-6-phosphate, phenylphosphate, p-aminophenylphosphate, p-aminophenyl-β-galactoside, urea or benzyl penicillin.

27. The device of claim 26 wherein the electrode comprises carbon, a metal, a metal oxide or a conductive polymeric material.

28. The device of claim 27 wherein the electrode comprises carbon paste, carbon fiber, graphite, glassy carbon, gold, silver, copper, platinum, palladium, indium tin oxide, poly(3,4-ethylenedioxythiophene) (PEDOT) or polyaniline.

29. The device of claim 28 wherein the redox polymer comprises quinone, ferrocene, osmium (4,4′-dimethyl-2,2′-bipyridine)₂,tetrathiafulvalene, an Ru complex, a Co complex, an Fe complex, or an Rh complex as a redox centre.

30. The device of claim 29 wherein the redox polymer comprises polyvinylpyridine, polysiloxane, polypyrrole, polyquinone or polyvinylpyridine-co-acrylamide.
